(19) United States
(12) Patent Application Publication
(10) Pub. No.: US 2009/0018142 A9
(43) Pub. Date: Jan. 15, 2009

(54) Use of phosphatases to treat tumors overexpressing N-CoR

(76) Inventors: Zhengping Zhuang, Bethesda, MD (US); Edward H. Oldfield, Philmont, VA (US); Deric M. Park, Pittsburgh, PA (US); Irina Lubensky, Potomac, MD (US); Jie Li, Gaithersburg, MD (US); John S. Kovach, East Setauket, NY (US)

Correspondence Address: COOPER & DUNHAM, LLP, 1185 AVENUE OF THE AMERICAS, NEW YORK, NY, 10036, US

(21) Appl. No.: 11/703,401

(22) Filed: Feb. 6, 2007

Related U.S. Application Data
(60) Provisional application No. 60/797,201, filed on May 2, 2006.

(51) Int. Cl. A61K 31/496; C12Q 1/02; G01N 33/50; A61P 35/00

(52) U.S. Cl. 514/255.01; 435/29; 436/86

(57) ABSTRACT

This invention provides a method of treating a patient suffering from a tumor overexpressing N—CoR comprising administering to the patient a phosphatase ligand, alone or in combination with a retinoid receptor ligand, a histone deacetylase ligand, or both, in amounts effective to treat the patient. This invention also provides a method of inhibiting tumor growth in a patient suffering from a tumor overexpressing N—CoR. This invention further provides a method of identifying a compound or a mixture of compounds capable of inducing differentiation of cells of a tumor overexpressing N—CoR. This invention still further provides a method of determining the likelihood of successfully treating a subject suffering from a tumor overexpressing N—CoR. This invention also provides a method of assessing the likelihood that a patient is suffering from a tumor overexpressing N—CoR. This invention yet also provides a method of assessing the likelihood that a patient previously suffering from and treated for a tumor overexpressing N—CoR has suffered a recurrence of a tumor overexpressing N—CoR. Finally, this invention provides analogous methods for use on glioblastoma multiforme.

The present application claims the benefit of U.S. Provisional Application No. 60/797,201, filed May 2, 2006 and U.S. Provisional Application 60/771,163, filed Feb. 6, 2006, the contents of each of which are incorporated by reference herein.

Certain embodiments of this invention were created in the performance of a Cooperative Research and Development Agreement with the National Institute of Health, United States Department of Health and Human Services. Consequently, the Government of the United States has certain rights in the invention.

Throughout this application, certain publications are referenced. Full citations for these publications may be found immediately preceding the claims. The disclosures of these publications in their entireties are hereby incorporated by reference into this application in order to more fully describe the state of the art to which this invention relates.

### BACKGROUND OF THE INVENTION

Despite the medical advances of the past few decades, cancer continues to plague people of all ages. The prevalence of various forms of cancer and the lack of effective treatments for many forms is a testament to the problems these diseases present.

Of the many cancers still lacking an effective treatment, glioblastoma multiforme (GBM) is one of the most lethal. Patients diagnosed with GBM have a grim prognosis. Patients may be treated with surgery, radiotherapy and chemotherapy, but the median survival is still less than one year. (Stupp et al. (2005)) This short survival time has remained virtually unchanged over the past 30 years due to the lack of an effective treatment.

GBM is the most common primary brain tumor. GBM is also the most malignant primary brain tumor. (Stupp et al. (2005)) It grows rapidly within the brain and may reach a large size before any symptoms occur and a diagnosis is made. GBM's malignancy typically remains in the cerebral hemispheres of the brain; however, glioblastomas can form in the brainstem, the cerebellum and the spinal cord. GBM does not usually spread to other parts of the body.

GBM tumors form from the supportive or glial tissue of the brain. GBM tumor cells look very different from normal brain cells. GBM cells are poorly differentiated, neoplastic astrocytes. GBM tumors are characterized by molecular lesions, cellular pleomorphism, mitotic figures, and multinucleated giant cells. (U.S. Patent Publication No. 2005/0203082, Hsu et al.) The World Health Organization classifies GBM as having 3 or 4 of the following histologic criteria: (1) nuclear atypia, (2) mitoses, (3) endothelial proliferation, and (4) necrosis.

The cause of GBM is unknown. GBM tumors may develop from much less malignant precursor tumors, called astrocytomas (secondary GBMs), or it may form de novo, with no evidence of a precursor tumor (primary GBM).

Due to the lethality of GBM and the sensitive nature of its location within the human body, it is imperative that new treatments and better modes of diagnosis be developed.

The subject invention provides novel methods of treating GBM. It also provides novel methods of diagnosing and screening for this deadly disease.

### SUMMARY OF THE INVENTION

The invention provides a method of treating a patient suffering from a tumor overexpressing N—CoR comprising administering to the patient one or more phosphatase ligand, alone or in combination with one or more retinoid receptor ligand, or one or more histone deacetylase ligand, or both, in each case in an amount effective to treat the patient.

This invention also provides a method of inhibiting growth of a tumor overexpressing N—CoR in a patient, comprising administering to the patient one or more phosphatase ligand, alone or in combination with one or more retinoid receptor ligand, or one or more histone deacetylase ligand, or both, in each case in amounts effective to affect N—CoR so as to induce differentiation of cells of the tumor overexpressing N—CoR and inhibit growth of the tumor in the patient.

This invention further provides a method of identifying a compound or a mixture of compounds capable of inducing differentiation or inhibiting proliferation of cells of a tumor overexpressing N—CoR, comprising the steps of (a) culturing a first population of the specified human cells in the absence of the compound or the mixture of compounds in both serum and serum free conditions; (b) separately culturing a second population of such human cells in the presence of the compound or the mixture of compounds; (c) comparing the rate of growth of the cultured human cells in step (a) with the rate of growth of the cultured human cells in step (b); (d) identifying the compound or the mixture of compounds which inhibited, or reduced the rate of, growth of the cultured human cells in step (b) as compared to the rate of growth of the cultured human cells in step (a); and (e) measuring the level of N—CoR in the cytoplasm and in the nucleus of the cultured human cells from step (b) whose growth was inhibited or whose rate of growth was reduced in the presence of the compound or the mixture of compounds with the levels of N—CoR in the cultured human cells from step (a), wherein the presence in the sample of decreased levels of N—CoR indicates that the compound or the mixture of compounds is capable of inducing differentiation of cells of tumors overexpressing N—CoR, so as to thereby identify the compound or the mixture of compounds.

This invention also provides a method of determining the likelihood of successfully treating a subject suffering from a tumor overexpressing N—CoR, comprising the steps of (a) obtaining a sample from the subject containing cells of a tumor overexpressing N—CoR; and (b) measuring the level of N—CoR in the cytoplasm and in the nucleus of cells in the sample so obtained, wherein the presence in the sample of an increased level of N—CoR in the nucleus of the cells indicates that there is a greater likelihood of successfully treating the subject.

This invention further provides a method of assessing the likelihood that a patient is suffering from a tumor overexpressing N—CoR, comprising the steps of (a) obtaining a serum sample from the subject; and (b) measuring the level of N—CoR in the serum sample so obtained, wherein the presence in the serum sample of increased levels of N—CoR relative to a normal reference standard indicates that the patient is likely suffering from a tumor overexpressing N—CoR.

This invention still further provides a method of assessing the likelihood that a patient previously suffering from and treated for a tumor overexpressing N—CoR has suffered a recurrence of such tumor, comprising the steps of (a) obtaining a serum sample from the subject; and (b) measuring the level of N—CoR in the serum sample so obtained, wherein the presence in the serum sample of increased levels of N—CoR relative to a previous level of N—CoR indicates that the patient is likely suffering from a recurrence of a tumor overexpressing N—CoR.

This invention provides a method of treating a patient suffering from glioblastoma multiforme, comprising administering to the patient one or more phosphatase ligand, alone or in combination with one or more retinoid receptor ligand, or one or more histone deacetylase ligand, or both, in each case in amounts effective to treat the patient.

This invention also provides a method of inhibiting growth of a tumor in a patient suffering from glioblastoma multiforme, comprising administering to the patient one or more phosphatase ligand, alone or in combination with one or more retinoid receptor ligand, or one or more histone deacetylase ligand, or both, in each case in amounts effective to affect N—CoR so as to induce differentiation of glioblastoma multiforme tumor cells and inhibit growth of the tumor in the patient.

This invention further provides a method of identifying a compound or a mixture of compounds capable of inducing differentiation or inhibiting proliferation of glioblastoma multiforme tumor cells, comprising the steps of (a) culturing a first population of human brain cells in the absence of the compound or the mixture of compounds in both serum and serum free conditions; (b) separately culturing a second population of such human brain cells in the presence of the compound or the mixture of compounds; (c) comparing the rate of growth of the cultured human brain cells in step (a) with the rate of growth of the cultured human brain cells in step (b); (d) identifying the compound or the mixture of compounds which inhibited, or reduced the rate of, growth of the cultured human brain cells in step (b) as compared to the rate of growth of the cultured human brain cells in step (a); and (e) measuring the level of N—CoR and the level of a glioblastoma multiforme lineage marker in the cytoplasm and in the nucleus of the cultured human brain cells from step (b) whose growth was inhibited or whose rate of growth was reduced in the presence of the compound or the mixture of compounds with the levels of N—CoR and the glioblastoma multiforme lineage marker in the cultured human brain cells from step (a), wherein a decrease in the level of N—CoR and an increase in the glioblastoma multiforme lineage marker indicate that the compound or the mixture of compounds is capable of inducing differentiation of glioblastoma multiforme tumor cells, so as to thereby identify the compound or the mixture of compounds.

This invention also provides a method of determining the likelihood of successfully treating a subject suffering from glioblastoma multiforme, comprising the steps of (a) obtaining a sample from the subject containing glioblastoma multiforme cells; and (b) measuring the level of each of N—CoR and a glioblastoma multiforme lineage marker in the cytoplasm and in the nucleus of cells in the sample so obtained, wherein the presence in the sample of an increased level of N—CoR and a low or undetectable level of glioblastoma multiforme lineage marker in the cytoplasm of the cells indicates that there is a greater likelihood of successfully treating the subject.

This invention still further provides a method of assessing the likelihood that a patient is suffering from glioblastoma multiforme, comprising the steps of (a) obtaining a sample of cerebrospinal fluid and/or tumor cells or serum from the subject; and (b) measuring the level of N—CoR in the cerebrospinal fluid and/or the cells or serum in the sample so obtained, wherein the presence in the sample of increased levels of N—CoR in the cerebrospinal fluid relative to a normal reference standard indicates that the patient is likely suffering from glioblastoma multiforme. If N—CoR is increased in the serum but not in the cerebral spinal fluid this would indicate that the patient is likely suffering from a tumor overexpressing N—CoR but not necessarily a glioblastoma multiforme.

Finally, this invention provides a method of assessing the likelihood that a patient previously suffering from and treated for glioblastoma multiforme has suffered a recurrence of glioblastoma multiforme, comprising the steps of (a) obtaining a sample of cerebrospinal fluid and/or tumor cells or serum from the subject; and (b) measuring the level of N—CoR in the cerebrospinal fluid and/or in the cells or serum in the sample so obtained, wherein the presence in the sample of increased levels of N—CoR in the cerebrospinal fluid or serum relative to the amount of N—CoR previously in the cerebral spinal fluid indicates that the patient is likely suffering from a recurrence of glioblastoma multiforme.

### BRIEF DESCRIPTION OF THE FIGURES

FIG. 1A. Differential expression of N—CoR in normal and GBM brain tissue. Total proteomic analysis of microdissected normal glial tissue (white matter) compared with GBM was performed by two dimensional gel electrophoresis (2-DGE). The highlighted region which is magnified on the bottom panels shows a consistent protein pattern in normal glial and GBM and unique expression of N—CoR in GBM. Protein identification was performed by liquid chromatography-mass spectrometry.

FIG. 1B. Expression of N—CoR in GBM by immunohistochemistry: N—CoR protein is present in both nucleus and cytoplasm in GBM. Right panel: arrows point to N—CoR staining (circled) in nucleus (right) and cytoplasm (left). Left panel: no N—CoR is seen in normal tissue.

FIG. 1C. Expression of N—CoR in GBM by Western blot analysis: N—CoR is present in GBMs on lanes 2-4 (molecular weight 270 kDa). N—CoR is absent in normal white matter (lane 1). β-actin was used as internal positive quantitative control.

FIG. 1D. Subcellular localization of N—CoR correlates with glial differentiation: N—CoR and GFAP immunolabeling in GBM. Cell on right demonstrating nuclear N—CoR localization shows no cytoplasmic GFAP. Cell on left with absent N—CoR labeling shows cytoplasmic expression of GFAP.

FIG. 1E. Co-expression of nuclear localization of N—CoR and cytoplasmic expression of CD133 in GBM primary culture: Immunolabeling of nuclear localization of N—CoR and cytoplasmic CD133 are present in the same GBM cells.

FIG. 2A. GFAP expression by CNTF-treated BTSC: GFAP expression was induced in glioma stem cells by treatment with CNTF and detected by 2-DGE and LCMS. Arrow points to the GFAP spot (circled).

FIG. 2B. Cytoplasmic N—CoR fraction increased by CNTF treatment of BTSC. Cytoplasmic level of N—CoR expression in BTSC shows gradual increase from day 0 to day 7 upon CNTF treatment. β-actin is shown as quantitative internal control.

FIG. 2C. Logarithmic growth curve of gliomal cell line, U343 MG-A, treated with retinoic acid (RA), okadaic acid (OA), combination of retinoic acid and okadaic acid (RA/OA), and control (NC) for 16 days: Individual treatment with retinoic acid and okadaic acid show a modest inhibition of growth. Combination of retinoic acid and okadaic acid shows synergistic reduction in cell growth. Error bars indicate 1 SD.

FIG. 3. Logarithmic curve of gliomal cell line U373 treated with endothal (End), endothal thioanhydride (ET), nor-cantharidin (nor-Can) and compound LB-1. Increasing dosages demonstrate a greater inhibition of growth. Error bars indicate SD.

FIG. 4A. Logarithmic curve of gliomal cell line U373 treated with all-trans retinoic acid (ATRA). Increasing dosage shows a modest inhibition of growth. Error bars indicate SD.

FIG. 4B. Inhibition of gliomal cell line U373 treated with endothal (End) and compound LB-1 with and without all-trans Retinoic acid (ATRA) for 7 days. Individual treatment with endothal and compound LB-1 shows modest inhibition of growth. Combination of End or compound LB-1 with ATRA shows synergistic reduction in cell growth. Error bars indicate SD.

FIG. 4C. Inhibition of growth of gliomal cell line U373 by endothal (End) with and without 13-cis Retinoic Acid (cis-RA). Individual treatment with End and cis-RA show a modest inhibition of growth. Combination of End and cis-RA show a synergistic reduction in cell growth. Error bars indicate SD.

FIG. 5A. Inhibition of growth of gliomal cell line U373 with Valproic Acid (Val). Increasing doses of Val (mM) shows a greater inhibition of cell growth. Error bars indicate SD.

FIG. 5B. Inhibition of growth of gliomal cell line U373 by Trichostatin A (TSA). Increasing doses of TSA (ug/mL) show a greater inhibition of cell growth. Error bars indicate SD.

FIG. 6A. Inhibition of growth of kidney cancer cell line, UMRC by endothal thioanhydride (ET), endothal (End), all-trans Retinoic Acid (ATRA), Trichostatin A (TSA) and norcantharidin (nor-Can) for 7 days. Error bars indicate SD

FIG. 6B. Inhibition of growth of gliomal cell line U373 by endothal thioanhydride (ET), endothal (End), all-trans Retinoic Acid (ATRA), Trichostatin A (TSA) and norcantharidin (nor-Can) for 7 days. Individual treatment with endothal thioanhydride showed the greatest inhibition of growth. Error bars indicate SD.

FIG. 6C. Inhibition of growth of breast cancer cell line, MCF-7 by Inhibition of UMRC by endothal thioanhydride (ET), endothal (End), all-trans Retinoic Acid (ATRA), Trichostatin A (TSA) and norcantharidin (nor-Can) for 7 days. Treatment with individual doses of endothal, ATRA and TSA surprisingly showed an inhibition in growth. Error bars indicate SD.

### DETAILED DESCRIPTION OF THE INVENTION

As used in this application each of the following terms has the meaning set forth below.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “therapeutically effective amount” means an amount sufficient to treat a subject afflicted with a disease (e.g. glioblastoma multiforme) or to alleviate a symptom or a complication associated with the disease.

As used herein, “treating” means slowing, stopping or reversing the progression of a disease, particularly glioblastoma multiforme.

As used herein, “overexpressing N—CoR” means that the level of the nuclear co-receptor (N—CoR) expressed in cells of the tissue tested are elevated in comparison to the levels of N—CoR as measured in normal healthy cells of the same type of tissue under analogous conditions. The nuclear receptor co-repressor (N—CoR) of the subject invention may be any molecule that binds to the ligand binding domain of the DNA-bound thyroid hormone receptor (T₃R) and retinoic acid receptor (RAR). (U.S. Pat. No. 6,949,624, Liu et al.) Examples of tumors that overexpress N—CoR may include glioblastoma multiforme, breast cancer (Myers et al.), colorectal cancer (Giannini and Cavallini), small cell lung cancer (Waters et al.) and ovarian cancer (Havrilesky et al.).

The invention provides a method of treating a patient suffering from a tumor overexpressing N—CoR comprising administering to the patient one or more phosphatase ligand, alone or in combination with one or more retinoid receptor ligand, or one or more histone deacetylase ligand, or both, in each case in an amount effective to treat the patient.

The phosphatase ligand may be selected from the group consisting of 1-nor-okadaone, antimonyl tartrate, bioallethrin, calcineurin, cantharidic acid, cantharidin, calyculin, cypermethrin, DARPP-32, deamidine, deltamethrin, diaminopyrroloquinazolines, endothal, endothal thioanhydride, fenvalerate, fostriecin, imidazoles, ketoconazole, L-4-bromotetramisole, levamisole, microcystin LA, microcystin LR, microcystin LW, microcystin RR, molybdate salts, okadaic acid, okadol, norcantharidin, pentamidine, pentavalent antimonials, permethrin, phenylarsine oxide, phloridzin, protein phosphatase inhibitor-1 (I-1), protein phosphatase inhibitor-2 (I-2)pyrophosphate, salubrinal, sodium fluoride, sodium orthovanadate, sodium stibogluconate, tartrate salts, tautomycin, tetramisole, thrysiferyl-23-acetate, vanadate, vanadium salts and antileishmaniasis compounds, including suramin and analogues thereof.

In the method of the invention, the histone deacetylase ligand may be an inhibitor, e.g. the histone deacetylase inhibitor HDAC-3 (histone deacetylase-3). The histone deacetylase ligand may also be selected from the group consisting of 2-amino-8-oxo-9,10-epoxy-decanoyl, 3-(4-aroyl-1H-pyrrol-2-yl)-N-hydroxy-2-propenamide, APHA Compound 8, apicidin, arginine butyrate, butyric acid, depsipeptide, depudecin, HDAC-3, m-carboxycinnamic acid bis-hydroxamide, N-(2-aminophenyl)-4-[N-(pyridin-3-ylmethoxycarbonyl)aminomethyl]benzamide, MS 275, oxamfiatin, phenylbutyrate, pyroxamide, scriptaid, sirtinol, sodium butyrate, suberic bishydroxamic acid, suberoylanilide hydroxamic acid, trichostatin A, trapoxin A, trapoxin B and valproic acid.

The subject application encompasses compounds which inhibit the enzyme histone deacetylase (HDAC). These HDAC enzymes posttranslationally modify histones (U.S. Patent Publication No. 2004/0197888, Armour et al.) Histones are groups of proteins which associate with DNA in eukaryotic cells to form compacted structures called chromatin. This compaction allows an enormous amount of DNA to be located within the nucleus of a eukaryotic cell, but the compact structure of chromatin restricts the access of transcription factors to the DNA. Acetylation of the histones decreases the compaction of the chromatin allowing transcription factors to bind to the DNA. Deacetylation, catalysed by histone deacetylases (HDACs), increases the compaction of chromatin, thereby reducing transcription factor accessibility to DNA. Therefore, inhibitors of histone deacetylases prevent the compaction of chromatin, allowing transcription factors to bind to DNA and increase expression of the genes.

This invention also provides a method of inhibiting growth of a tumor overexpressing N—CoR in a patient, comprising administering to the patient one or more phosphatase ligand, alone or in combination with one or more retinoid receptor ligand, one or more histone deacetylase ligand, or both, in each case in amounts effective to affect N—CoR so as to thereby induce differentiation of cells of the tumor overexpressing N—CoR and inhibit growth of the tumor in the patient.

This invention further provides a method of identifying a compound or a mixture of compounds capable of inducing differentiation or inhibiting proliferation of cells of a tumor overexpressing N—CoR, comprising the steps of (a) culturing a first population of the specified human cells in the absence of the compound or the mixture of compounds in both serum and serum free conditions; (b) separately culturing a second population of such human cells in the presence of the compound or the mixture of compounds; (c) comparing the rate of growth of the cultured human cells in step (a) with the rate of growth of the cultured human cells in step (b); (d) identifying the compound or the mixture of compounds which inhibited, or reduced the rate of, growth of the cultured human cells in step (b) as compared to the rate of growth of the cultured human cells in step (a); and (e) measuring the level of N—CoR in the cytoplasm and in the nucleus of the cultured human cells from step (b) whose growth was inhibited or whose rate of growth was reduced in the presence of the compound or the mixture of compounds with the levels of N—CoR in the cultured human cells from step (a), wherein the presence in the sample of decreased levels of N—CoR indicates that the compound or the mixture of compounds is capable of inducing differentiation of cells of tumors overexpressing N—CoR, so as to thereby identify the compound or the mixture of compounds.

In the method of the invention, the level of N—CoR in the cytoplasm and in the nucleus may be measured by either indirect immunofluorescence microscopy, direct immunofluorescence microscopy, FACS, or other methods for detecting and measuring amounts of specific proteins in tissues including assessment of the amounts of proteins in the nucleus versus the cytoplasm and in cell lysates, or a combination thereof.

N—CoR is expressed in the nucleus of the undifferentiated tumor or stem cells and is only present in the cytoplasm at amounts detectable by immunochemistry and Western blotting when the cell undergoes differentiation. N—CoR is not detectable by immunochemistry and Western blotting in either the nucleus or the cytoplasm of normal or fully differentiated cells.

Thus, in the methods of the invention, an assessment of the percentage of cells with N—CoR in the cytoplasm relative to the percentage of cells with N—CoR in the nucleus is representative of the ratio of more differentiated cells to less differentiated cells in a given tissue.

In the method of the invention, tumors that overexpress N—CoR may include glioblastoma multiforme, breast cancer, colorectal cancer, small cell lung cancer and ovarian cancer.

This invention also provides a method of determining the likelihood of successfully treating a subject suffering from a tumor overexpressing N—CoR, comprising the steps of (a) obtaining a sample from the subject containing cells of a tumor overexpressing N—CoR; and (b) measuring the level of N—CoR in the cytoplasm and in the nucleus of cells in the sample so obtained, wherein the presence in the sample of an increased level of N—CoR in the nucleus of the cells indicates that there is a greater likelihood of successfully treating the subject.

In the method, the level of N—CoR in the cytoplasm and in the nucleus may be measured by either indirect immunofluorescence microscopy, direct immunofluorescence microscopy, FACS, or other methods for detecting and measuring amounts of specific proteins in tissues including assessment of the amounts of proteins in the nucleus versus the cytoplasm and in cell lysates, or a combination thereof.

N—CoR is expressed in the nucleus of the undifferentiated tumor or stem cells and is only present in the cytoplasm at amounts detectable by immunochemistry and Western blotting when the cell undergoes differentiation. N—CoR is not detectable by immunochemistry and Western blotting in either the nucleus or the cytoplasm of normal or fully differentiated cells.

Thus, in the methods of the invention, an assessment of the percentage of cells with N—CoR in the cytoplasm relative to the percentage of cells with N—CoR in the nucleus is representative of the ratio of more differentiated cells to less differentiated cells in a given tissue.

In the method of the invention, tumors that overexpress N—CoR may include glioblastoma multiforme, breast cancer, colorectal cancer, small cell lung cancer and ovarian cancer.

This invention further provides a method of assessing the likelihood that a patient is suffering from a tumor overexpressing N—CoR, comprising the steps of (a) obtaining a serum sample from the subject; and (b) measuring the level of N—CoR in the serum sample so obtained, wherein the presence in the serum sample of increased levels of N—CoR relative to a normal reference standard indicates that the patient is likely suffering from a tumor overexpressing N—CoR.

In the method of the invention, tumors that overexpress N—CoR may include glioblastoma multiforme, breast cancer, colorectal cancer, small cell lung cancer and ovarian cancer.

This invention still further provides a method of assessing the likelihood that a patient previously suffering from and treated for a tumor overexpressing N—CoR has suffered a recurrence of such tumor, comprising the steps of (a) obtaining a serum sample from the subject; and (b) measuring the level of N—CoR in the serum sample so obtained, wherein the presence in the serum sample of increased levels of N—CoR relative to a previously lower level indicates that the patient is likely suffering from a recurrence of a tumor overexpressing N—CoR.

In the method of the invention, tumors that overexpress N—CoR may include glioblastoma multiforme, breast cancer, colorectal cancer, small cell lung cancer and ovarian cancer.

This invention provides a method of treating a patient suffering from glioblastoma multiforme, comprising administering to the patient one or more phosphatase ligand, alone or in combination with one or more retinoid receptor ligand, or one or more histone deacetylase ligand, or both, in each case in amounts effective to treat the patient.

The phosphatase ligand may be selected from the group consisting of 1-nor-okadaone, antimonyl tartrate, bioallethrin, calcineurin, cantharidic acid, cantharidin, calyculin, cypermethrin, DARPP-32, deamidine, deltamethrin, diaminopyrroloquinazolines, endothal, endothal thioanhydride, fenvalerate, fostriecin, imidazoles, ketoconazole, L-4-bromotetramisole, levamisole, 1-p-bromotetramisole, d-p-bromotetramisole, p-hydroxylevamisole, microcystin LA, microcystin LR, microcystin LW, microcystin RR, molybdate salts, okadaic acid, okadol, norcantharidin, pentamidine, pentavalent antimonials, permethrin, phenylarsine oxide, phloridzin, protein phosphatase inhibitor-1 (I-1), protein phosphatase inhibitor-2 (I-2)pyrophosphate, salubrinal, sodium fluoride, sodium orthovanadate, sodium stibogluconate, tartrate salts, tautomycin, tetramisole, thrysiferyl-23-acetate, vanadate, vanadium salts and antileishmaniasis compounds, including suramin and analogues thereof.

In a presently preferred embodiment of the invention, the phosphatase ligand is a protein phosphatase inhibitor, such as endothal thioanhydride, endothal, norcantharidin or okadaic acid.

The protein phosphatases of the subject application can be tyrosine-specific, serine/threonine-specific, dual-specificity phosphatases, alkaline phosphatases such as levamisole, and acid phosphatases.

In the method of the invention, the retinoid receptor ligand may be a retinoid, such as a retinoic acid, e.g. cis retinoic acid or trans retinoic acid. The cis retinoic acid may be 13-cis retinoic acid and the trans retinoic acid may be all-trans retinoic acid.

In the practice of the method of the invention, the retinoid receptor ligand may affect retinoid receptor activity but not thyroid hormone receptor activity; alternatively or additionally the retinoid receptor ligand may inhibit N—CoR binding to the retinoid receptor but not N—CoR binding to the thyroid hormone receptor.

Retinoid receptor ligands used in the method of the invention include vitamin A (retinol) and all its natural and synthetic derivatives (retinoids).

In the method of the invention, the retinoid receptor ligand may be selected from the group consisting of b,g-selective 6-(5,6,7,8-tetrahydro-5,5,8,8-tetramethyl-2-naphthalenyl)-2-naph-thalenecarboxylic acid (TTNN), Z-oxime of 6-(5,6,7,8-tetrahydro-5,5,8,8-tetramethyl-2-naphthalenylcarbonyl)-2-naphthalenecarboxylic acid (SR11254), 4-(5,6,7,8-tetrahydro-5,5,8,8-tetramethyl-2-anthracenyl)benzoic acid (TTAB), 4-[1-(5,6,7,8-tetrahydro-5,5,8,8-tetramethyl-2-naphthalenyl)-cyclopropyl]benzoic acid (SR11246), 4-[1-(5,6,7,8-tetrahydro-3,5,5,8,8-pentamethyl-2-naphthalenyl)-2-methylpropenyl]benzoic acid (SR11345), and 2-(6-carboxy-2-naphthalenyl)-2-(5,6,7,8-tetrahydro-5,5,8,8-tetramethyl-2-naphthalenyl)-1,3-dithiolane (SR11253).

In the method of the invention, the histone deacetylase ligand may be an inhibitor, e.g. the histone deacetylase inhibitor HDAC-3 (histone deacetylase-3). The histone deacetylase ligand may also be selected from the group consisting of 2-amino-8-oxo-9,10-epoxy-decanoyl, 3-(4-aroyl-1H-pyrrol-2-yl)-N-hydroxy-2-propenamide, APHA Compound 8, apicidin, arginine butyrate, butyric acid, depsipeptide, depudecin, HDAC-3, m-carboxycinnamic acid bis-hydroxamide, N-(2-aminophenyl)-4-[N-(pyridin-3-ylmethoxycarbonyl)aminomethyl]benzamide, MS 275, oxamfiatin, phenylbutyrate, pyroxamide, scriptaid, sirtinol, sodium butyrate, suberic bishydroxamic acid, suberoylanilide hydroxamic acid, trichostatin A, trapoxin A, trapoxin B and valproic acid. The subject application encompasses compounds which inhibit the enzyme histone deacetylase (HDAC). These HDAC enzymes posttranslationally modify histones (U.S. Patent Publication No. 2004/0197888, Armour et al.) Histones are groups of proteins which associate with DNA in eukaryotic cells to form compacted structures called chromatin. This compaction allows an enormous amount of DNA to be located within the nucleus of a eukaryotic cell, but the compact structure of chromatin restricts the access of transcription factors to the DNA. Acetylation of the histones decreases the compaction of the chromatin allowing transcription factors to bind to the DNA. Deacetylation, catalysed by histone deacetylases (HDACs), increases the compaction of chromatin, thereby reducing transcription factor accessibility to DNA. Therefore, inhibitors of histone deacetylases prevent the compaction of chromatin, allowing transcription factors to bind to DNA and increase expression of the genes.

This invention also provides a method of inhibiting growth of a tumor in a patient suffering from glioblastoma multiforme, comprising administering to the patient one or more phosphatase ligand, alone or in combination with one or more retinoid receptor ligand, one or more histone deacetylase ligand, or both, in each case in amounts effective to affect N—CoR so as to thereby induce differentiation of glioblastoma multiforme tumor cells and inhibit growth of the tumor in the patient.

The nuclear receptor co-repressor (N—CoR) of the subject invention may be any molecule that binds to the ligand binding domain of the DNA-bound thyroid hormone receptor (T₃R) and retinoic acid receptor (RAR). (U.S. Pat. No. 6,949,624, Liu et al.)

This invention further provides a method of identifying a compound or a mixture of compounds capable of inducing differentiation or inhibiting proliferation of glioblastoma multiforme tumor cells, comprising the steps of (a) culturing a first population of human brain cells in the absence of the compound or the mixture of compounds in both serum and serum free conditions; (b) separately culturing a second population of such human brain cells in the presence of the compound or the mixture of compounds; (c) comparing the rate of growth of the cultured human brain cells in step (a) with the rate of growth of the cultured human brain cells in step (b); (d) identifying the compound or the mixture of compounds which inhibited, or reduced the rate of, growth of the cultured human brain cells in step (b) as compared to the rate of growth of the cultured human brain cells in step (a); and (e) measuring the level of N—CoR and the level of a glioblastoma multiforme lineage marker in the cytoplasm and in the nucleus of the cultured human brain cells from step (b) whose growth was inhibited or whose rate of growth was reduced in the presence of the compound or the mixture of compounds with the levels of N—CoR and the glioblastoma multiforme lineage marker in the cultured human brain cells from step (a), wherein a decrease in the level of N-Cor and an increase in glioblastoma multiforme lineage marker indicate that the compound or the mixture of compounds is capable of inducing differentiation of glioblastoma multiforme tumor cells, so as to thereby identify the compound or the mixture of compounds.

In this method, the glioblastoma multiforme lineage marker may be selected from the group consisting of GFAP, nestin, tujl, and CNPase.

A glial fibrillary acidic protein (GFAP) useful in the subject invention is a 55 kDa cytosolic protein, a major structural component of astroglial filaments and the major intermediate filament protein in astrocytes. (U.S. Patent Publication No. 2004/0253637, Buechler et al.) GFAP is specific to astrocytes of the brain.

In the method of the invention, the level of N—CoR and the level of the glioblastoma multiforme lineage marker in the cytoplasm and in the nucleus may be measured by either indirect immunofluorescence microscopy, direct immunofluorescence microscopy, FACS, or other methods for detecting and measuring amounts of specific proteins in tissues including assessment of the amounts of proteins in the nucleus versus the cytoplasm and in cell lysates, or a combination thereof.

N—CoR is expressed in the nucleus of the undifferentiated tumor or stem cells and is only present in the cytoplasm at amounts detectable by immunochemistry and Western blotting when the cell undergoes differentiation. N—CoR is not detectable by immunochemistry and Western blotting in either the nucleus or the cytoplasm of normal or fully differentiated cells.

Thus, in the methods of the invention, an assessment of the percentage of cells with N—CoR in the cytoplasm relative to the percentage of cells with N—CoR in the nucleus is representative of the ratio of more differentiated cells to less differentiated cells in a given tissue. In the method, the first population of human brain cells and the second population of human brain cells is selected from the group consisting of primary normal human brain cells, primary human brain stem cells, and primary glioblastoma multiforme stem cells. For example, the first population of human brain cells and the second population of human brain cells may be the same or different, preferably the same and may be cells derived from any of the following cell lines: U343 MG-A, U251, U373, U87, A-172, LN-18, LN-229, M059J, M059K, and HS683.

Cell line U343 MG-A is available from the University of California at San Francisco (UCSF) Brain Tumor Research Center Tissue Bank. (University of California, San Francisco, Health Sciences West building, San Francisco, Calif. 94143-0520.) In addition, cell lines U343 and U87 are commercially available from EPO-GmbH, Robert-Rössle-Str. 10, 13092 Berlin-Buch, Germany.

Cell line U251 is available from Division of Cancer Treatment and Diagnosis at National Cancer Institute Tumor Repository, The National Cancer Institute at Frederick Bldg. 1073, Frederick, Md. 21702-1201.

Cell lines A-172, LN-18, LN-229, M059J, M059K, and HS683 are available from the American Type Culture Collection (ATCC), P.O. Box 1549, Manassas, Va., 20108, as ATCC No. CRL-1620, ATCC No. CRL-2610, ATCC No. CRL-229, ATCC No. CRL-2365 and ATCC No. HTB-138, respectively.

Cell line U373 is available from the National. Institute of Neurological Disease and Stroke, Building 31, 31 Center Drive, Bethesda, Md., 20892 and the National Institute of Health, Building 1, 1 Center Drive, Bethesda, Md., 20892.

This invention also provides a method of determining the likelihood of successfully treating a subject suffering from glioblastoma multiforme, comprising the steps of (a) obtaining a sample from the subject containing glioblastoma multiforme cells; and (b) measuring the level of each of N—CoR and a glioblastoma multiforme lineage marker in the cytoplasm and in the nucleus of cells in the sample so obtained, wherein the presence in the sample of an increased level of N—CoR in the nucleus indicated that there is a greater likelihood of successfully treating the subject.

In the preceding method, the glioblastoma multiforme lineage marker may be selected from the group consisting of GFAP, nestin, tujl, and CNPase.

In the method, the level of N—CoR and the level of the glioblastoma multiforme lineage marker in the cytoplasm and in the nucleus may be measured by either indirect immunofluorescence microscopy, direct immunofluorescence microscopy, FACS, or other methods for detecting and measuring amounts of specific proteins in tissues including assessment of the amounts of proteins in the nucleus versus the cytoplasm and in cell lysates, or a combination thereof.

N—CoR is expressed in the nucleus of the undifferentiated tumor or stem cells and is only present in the cytoplasm at amounts detectable by immunochemistry and Western blotting when the cell undergoes differentiation. N—CoR is not detectable by immunochemistry and Western blotting in either the nucleus or the cytoplasm of normal or fully differentiated cells.

Thus, in the methods of the invention, an assessment of the percentage of cells with N—CoR in the cytoplasm relative to the percentage of cells with N—CoR in the nucleus is representative of the ratio of more differentiated cells to less differentiated cells in a given tissue.

This invention also provides a method of assessing the likelihood that a patient is suffering from glioblastoma multiforme, comprising the steps of (a) obtaining a sample of cerebrospinal fluid and/or tumor cells from the subject; and (b) measuring the level of N—CoR in the cerebrospinal fluid and/or the cells in the sample so obtained, wherein the presence in the sample of increased levels of N—CoR in the cerebrospinal fluid relative to a normal reference standard indicates that the patient is likely suffering from glioblastoma multiforme. If N—CoR is increased in the serum but not in the cerebral spinal fluid this would indicate that the patient is likely suffering from a tumor overexpressing N—CoR but not necessarily a glioblastoma multiforme.

This invention also provides a method of assessing the likelihood that a patient previously suffering from and treated for glioblastoma multiforme has suffered a recurrence of glioblastoma multiforme, comprising the steps of (a) obtaining a sample of cerebrospinal fluid and/or tumor cells from the subject; and (b) measuring the level of N—CoR in the cerebrospinal fluid and/or the cells in the sample so obtained, wherein the presence in the sample of increased levels of N—CoR in the cerebrospinal fluid relative to the previous levels of N—CoR post-treatment indicates that the patient is likely suffering from a recurrence of glioblastoma multiforme.

This invention further provides a method of assessing the likelihood that a patient is suffering from a tumor overexpressing N—CoR, comprising the steps of (a) obtaining a serum sample from the subject; and (b) measuring the level of N—CoR in the serum sample so obtained, wherein the presence in the serum sample of increased levels of N—CoR relative to a normal reference standard indicates that the patient is likely suffering from a tumor overexpressing N—CoR.

This invention still further provides a method of assessing the likelihood that a patient previously suffering from and treated for a tumor overexpressing N—CoR has suffered a recurrence of a tumor overexpressing N—CoR, comprising the steps of (a) obtaining a serum sample from the subject; and (b) measuring the level of N—CoR in the serum sample so obtained, wherein the presence in the serum sample of increased levels of N—CoR relative to a previous lower levels of N—CoR post treatment indicates that the patient is likely suffering from a recurrence of a tumor overexpressing N—CoR.

The invention provides a use of one or more phosphatase ligand, in an amount effective to treat a patient, alone or in combination with one or more retinoid receptor ligand, or one or more histone deacetylase ligand, or both, in each case in an amount effective to treat the patient, for the preparation of a medicament. In an embodiment of the invention, the medicament comprises one or more phosphatase ligand alone, or for use with, one or more retinoid receptor ligand, or one or more histone deacetylase receptor ligand, or both.

This invention also provides the use of a phosphatase ligand, in an amount effective to induce differentiation of cells of a tumor overexpressing N—CoR and to inhibit the growth of the tumor in a patient, alone or in combination with one or more retinoid receptor ligand, one or more histone deacetylase ligand, or both, for the preparation of a medicament. In an embodiment of the invention, the medicament comprises one or more phosphatase ligand alone, or for use with, one or more retinoid receptor ligand, or one or more histone deacetylase receptor ligand, or both.

The uses of the invention herein encompass the enumerated phosphatase ligands, retinoid receptors and histone deacetylase receptor ligands enumerated above.

The invention provides a product containing a phosphatase ligand in combination with one or more retinoid receptor ligand, one or more histone deacetylase ligand, or both, as a combined preparation for simultaneous, separate or sequential use in treating a tumor overexpressing N—CoR.

This invention is illustrated in the Experimental Details section which follows. This section is set forth to aid in an understanding of the invention but is not intended to, and should not be construed to limit in any way the invention as set forth in the claims which follow thereafter.

### Experimental Details

### Materials and Methods

### EXAMPLE 1

To identify novel therapeutic targets for the treatment of glioblastoma multiforme, the proteomes of 7 GBM tissues and 7 normal brain tissues (white matter) were compared using selective microdissection, two dimensional gel electrophoresis (2-DGE) and liquid chromatography-mass spectroscopy (LCMS).

GBM tissue was further tested by immunohistochemistry and Western blotting for the expression of nuclear receptor co-repressor (N—CoR). β-actin was used as internal positive quantitative control for the Western blotting.

Expression of glial fibrillary acidic protein (GFAP), an established marker of astroglial differentiation and the subcellular localization of N—CoR was assessed by indirect immunofluorescence microscopy was on primary cell cultures, established cell lines (A-172, HS683, U87, U251, and U343 MG-A), frozen and paraffin embedded tissue sections of GBM. The GBM cell lines were all cultured in DMEM with 10% FCS and high glucose DMEM/F12 with N2 supplement (serum-free) on poly-1-ornithine and fibronectin coated plate/dish/flask.

To examine the role of N—CoR in GBM development and differentiation, cultured brain tumor stem cells (BTSC) isolated from GBM were treated with ciliary neurotrophic factor (CNTF), an agent which has previously been shown to induce the astrocytic differentiation of neural stem cells (NSC) in vitro. (Hughes et al. (1988)) The BTSCs cells were then assessed for GFAP expression along with subcellular N—CoR localization.

Finally, the GBM cell line U343 MG-A was treated with 50 μM of retinoic acid (RA) and/or 10 nM of okadaic acid (OA), a protein phosphatase-1 inhibitor.

Three cultures for each of four treatments were grown for 16 days with cell counts obtained at baseline and for each of the eight even numbered days. Log transformations were applied to the cell counts, and repeated measures of analysis of variance model used to evaluate the treatment differences over time. Pair wise treatment comparisons used Sidak's statistic to account for multiple testing.

### Results

Comparative proteomic analysis of glioblastoma multiforme (GBM) tissue and matched normal glial tissue demonstrated increased expression of the nuclear receptor co-repressor (N—CoR) in GBM. GBM tumor cells with nuclear localization of N—CoR were relatively undifferentiated, but subject to differentiation upon exposure to agents promoting phosphorylation of N—CoR and its translocation to the cytoplasm.

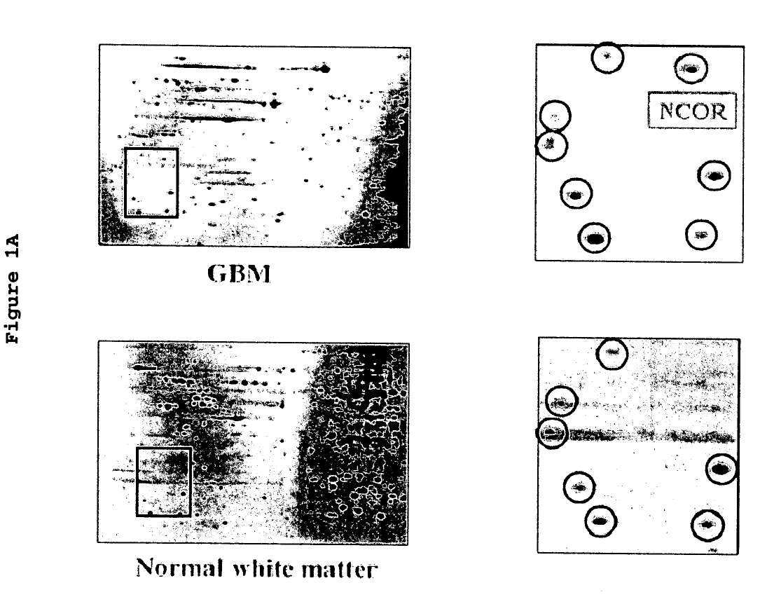

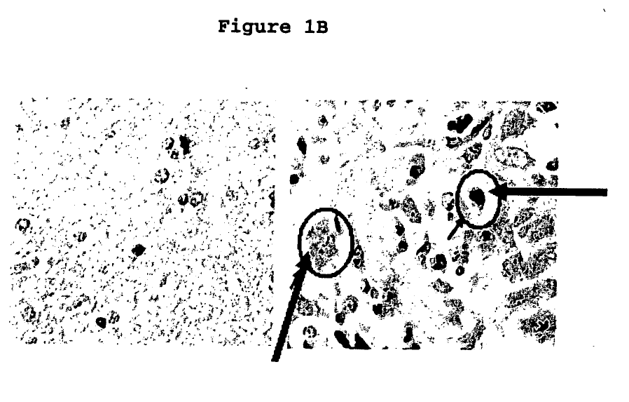

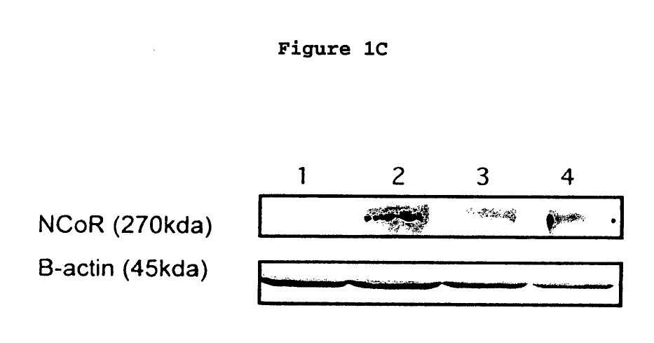

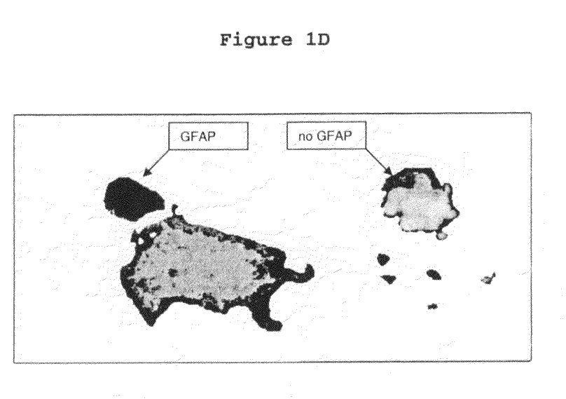

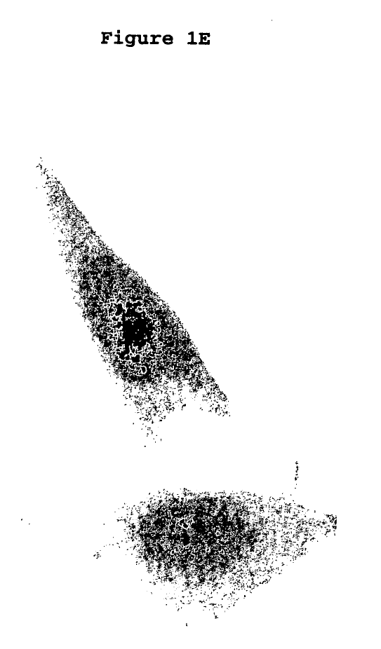

As shown in FIG. 1A, total proteomic analysis of microdissected normal glial tissue (white matter) compared with GBM shows a consistent protein pattern in normal white matter and GBM, and a unique expression of N—CoR in GBM. This expression of N—CoR in GBM tissue was confirmed by immunohistochemistry and Western blotting as shown in FIGS. 1B and 1C. In contrast, N—CoR was not detectable in normal brain topographically matched to the location of the tumor specimen.

As shown in FIG. 1D, Nuclear expression of N—CoR correlated with the absence of GFAP expression in the cells of primary cultures and tissue sections, whereas cytoplasmic expression of N—CoR correlated with positive expression of GFAP. Subcellular localization of N—CoR correlates with glial differentiation. Some of the tumor cells with nuclear expression of N—CoR also expressed CD133 as shown in FIG. 1E.

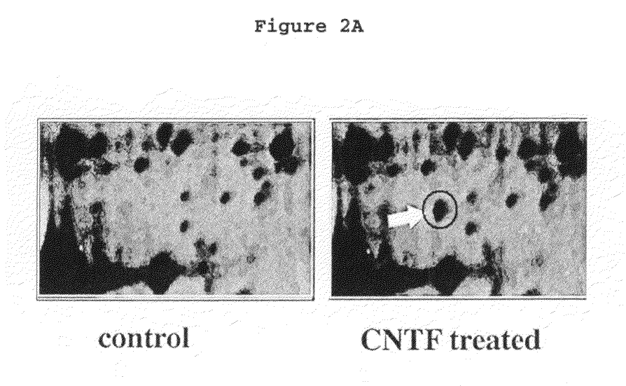

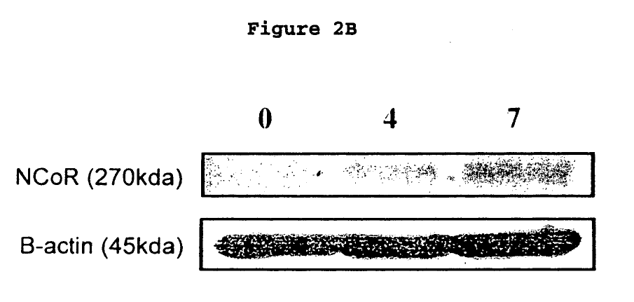

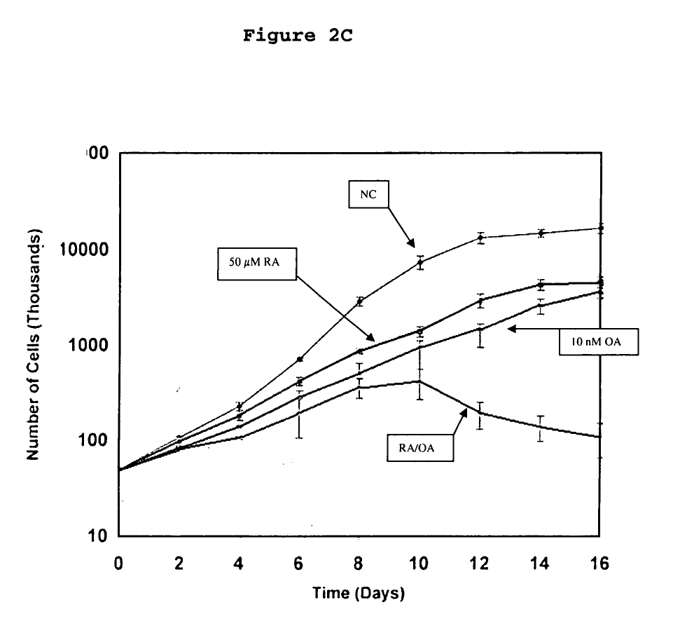

As shown in FIG. 2A, brain tumor stem cells (BTSCs) cultured with CNTF began to express GFAP. Western blot analysis of the cytoplasmic fraction of CNTF-treated BTSCs demonstrates the translocation of N—CoR to the cytoplasm (See FIG. 2B). Both cytoplasmic N—CoR and GFAP expression peaked at day 7 following CNTF stimulation.

FIG. 2C shows the logarithmic growth curve of gliomal cell line, U343 MG-A, treated with retinoic acid (RA), okadaic acid (OA), combination of retinoic acid and okadaic acid (RA/OA), and control (NC) for 16 days. Curve fitting indicated exponential cell count growth for each treatment except for the RA/OA treatment group. Analysis of variance models were used to examine differences across the three cultures for each treatment. For each treatment group the cultures were similar. Model based F-statistics indicated significant differences across time (p=0.006) and for time by treatment interactions (p=0.001). The differences in log cell counts among the four treatments were significant with an F_{3,8}=163.2 and the resulting p-value less than 0.0001. All pair wise treatment differences (Sidak's test) were significant with p-values less than 0.0001, except the RA vs OA difference (p=0.079). Cell counts monotonically increased for OA, RA, and controls through day 16. The number of cells with the combination treatment (OA+RA) increased until day 10 and then decreased for the remaining duration of the study. Retinoic acid and low-dose okadaic acid each had a modest effect on cell growth. Combination of retinoic acid and okadaic acid shows synergistic reduction in cell growth.

### EXAMPLE 2

### Effect of Cantharidin Analogs on GBM Cells

To identify novel therapeutic targets for the treatment of glioblastoma multiforme (GBM), cantharidin analogs were evaluated for their ability to inhibit growth of glioblastoma multiforme cells. Specifically, GBM cell line U373 was used in evaluations.

The cantharidin homologs that were evaluated were norcantharidin (nor-Can), which is a demethylated cantharidin; endothal (End), which is a dicarboxylic acid derivative of norcantharidin; endothal thioanhydride (ET); and the compound LB-1, which was obtained from Lixte Biotechnology, Inc., 248 Route 25A, No. 2, East Setauket, N.Y., which has the structure:

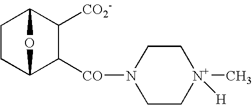

Cells were plated in triplicate on day one with and without different amounts of each drug dissolved in media (compound LB-1 and endothal) or in dimethylsulfoxide (endothal thioanhydride and norcantharidin). The total number of cells is counted in the triplicate cultures at each dose and in controls after 7 days and the average number of cells and the standard deviation is determined.

The amount of inhibition of GBM cell growth is expressed as the proportion of the number of cells in the experimental dishes compared to the number of cells in control dishes containing only the drug vehicle and culture medium. The average percent of control is plotted and bracketed by one standard deviation calculated from the triplicate measurements.

### Results

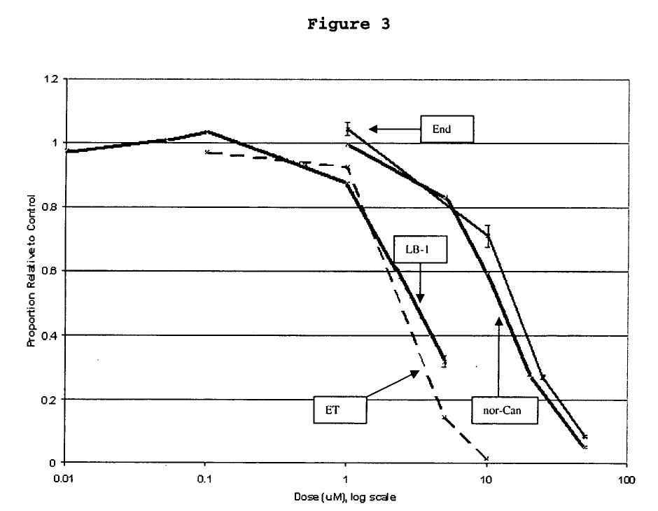

Each of the norcantharidin analogs inhibited the growth of GBMs in a dose dependent manner in vivo as shown in FIG. 3.

From graphic plots of the GBM cell line U373 as a function of exposure to different doses of drug for 7 days, the concentration of each compound that inhibited brain tumor cell proliferation by 50% (IC50) was estimated. The IC50s expressed in micro-molarity (uM), were: 2.5, 3.0, 12.0, and 15.0 for endothal thioanhydride, compound LB-1, norcantharidin, and endothal respectively as seen in FIG. 3.

### EXAMPLE 3

### Effect of Selected Cantharidin Analogs Combined with Retinoic Acid

To identify the effect of combinations of PP2A anti-phosphatases and retinoids affecting nuclear complexes, we focused on water soluble cantharidin derivatives that have been shown to be active against human GBMs in vitro, endothal and compound LB-1.

To observe the effects of endothal in combination with retinoic acids, endothal was combined with all-trans retinoic acid and 13-cis retinoic acid.

Cells were plated in triplicate on day one with and without different amounts of each drug dissolved in media (compound LB-1 and endothal). The total number of cells is counted in the triplicate cultures at each dose and in controls after 7 days and the average number of cells and the standard deviation is determined.

The amount of inhibition of GBM cell growth is expressed as the proportion of the number of cells in the experimental dishes compared to the number of cells in control dishes containing only the drug vehicle and culture medium. The average percent of control is plotted and bracketed by one standard deviation calculated from the triplicate measurements.

### Results

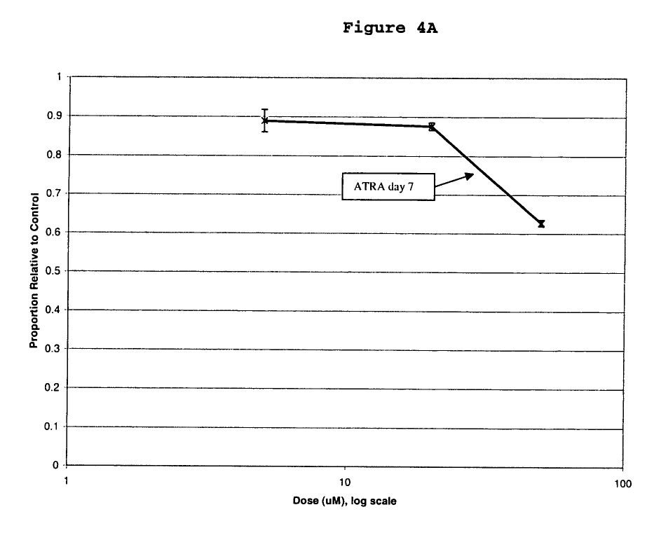

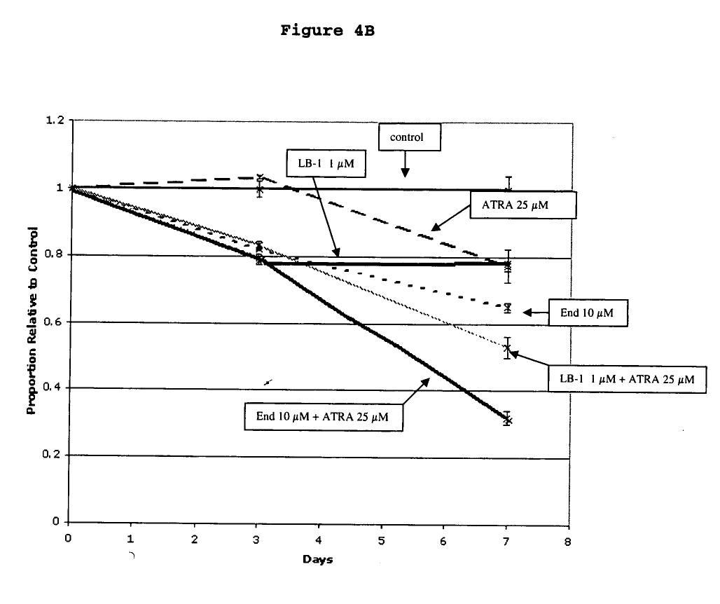

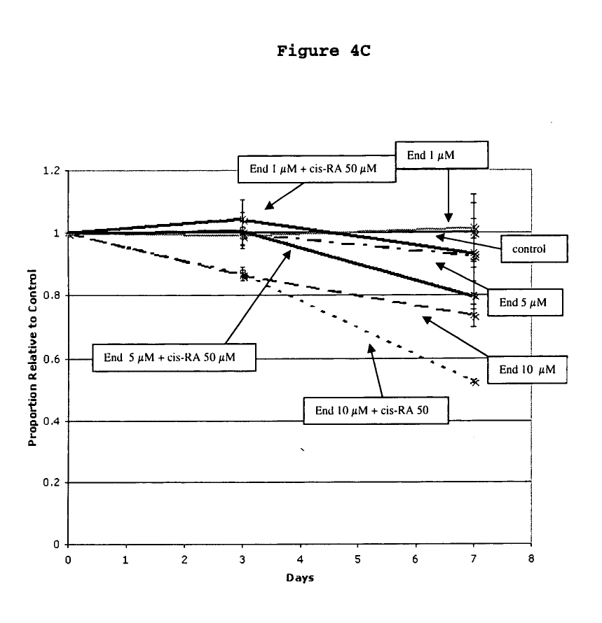

FIG. 4A demonstrates the effect of all-trans retinoic acid (ATRA) when used individually. The IC50 of ATRA alone was greater than 50 μM. Endothal and compound LB-1, each in combination with ATRA, synergistically inhibited proliferation of GBM cell line U373 as seen in FIG. 4B. Synergism (potentiation) of the inhibitory activity of two drugs in combination is said to be present when the percent survival in the presence of two drugs is less than the product of the percent survivals of the two drugs used alone at the same doses in the combination. The extent of synergism of compound LB-1 and endothal (end) in combination with ATRA is quantified below in Table 1:

TABLE 1
Endothal and compound LB-1 +/− ATRA
Inhibition of U373 Cells.

Percent of Control

| | | Expected if |
|---|---|---|
| | Observed | Additive |
| ATRA 25 uM | 77% | — |
| END 10 uM | 65% | — |
| ATRA 25 uM + END 10 uM | 32% | 50% |
| LB-1 1 uM | 78% | — |
| ATRA 25 uM + LB-1 1 uM | 53% | 60% |

The expected percent survival of U373 cells exposed to the combination of ATRA and End is 50% (77% by ATRA×65% by End=50%) whereas the observed survival was 32%. The expected percent survival in the presence of the combination of ATRA and LB-1 is 60% (77% by ATRA×78% by LB-1=60%), whereas the observed survival was 53%.

Endothal combined with 13-cis retinoic acid (cis-RA) synergistically inhibits U373 cell growth as seen in FIG. 4C. The extent of synergism is quantified below in Table 2.

TABLE 2
Endothal +/− 13-Cis RA: Inhibition of U373 Cells

13-Cis Retinoic Acid

| | |
|---|---|
| None | 50 uM |

| | |
|---|---|
| Endothal (μM) | Percent of Control |

| | | |
|---|---|---|
| 1 μM | 100% | 94% |
| 5 μM | 96% | 80% |
| 10 μM | 73% | 53% |

The presence of 13-cis retinoic acid at 50 μM had little effect when combined with endothal at 1.0 μM (96%). At higher doses of endothal, simultaneous exposure to the same amount of 13-cis retinoic acid decreased cell survival from 96% at 5 uM endothal alone to 80% in combination with 13-cis retinoic acid and from 76% survival at 10 uM endothal alone to 53% in combination with 13-cis retinoic acid.

### EXAMPLE 4

### Effect of Histone Deacetylase Ligands on U373 GBMS

Because retinoids are known to produce developmental abnormalities in the fetus when the drug is given to pregnant women, we studied the activity of valproic acid and Trichostatin A, drugs with low toxicity in the adult, but which also disrupt fetal development.

### Results:

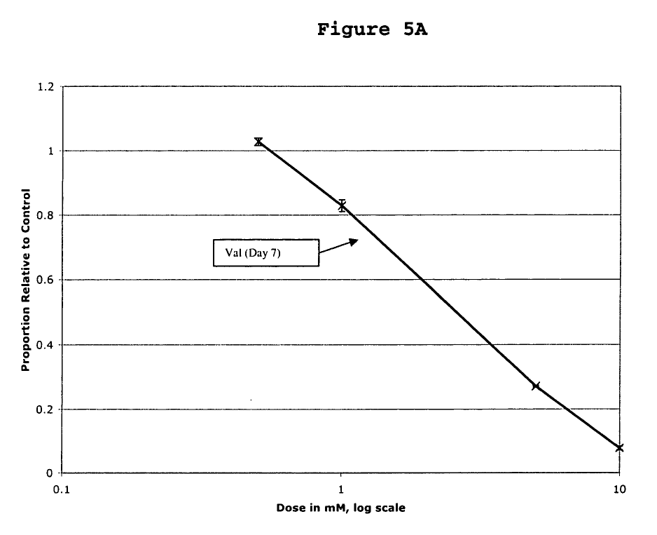

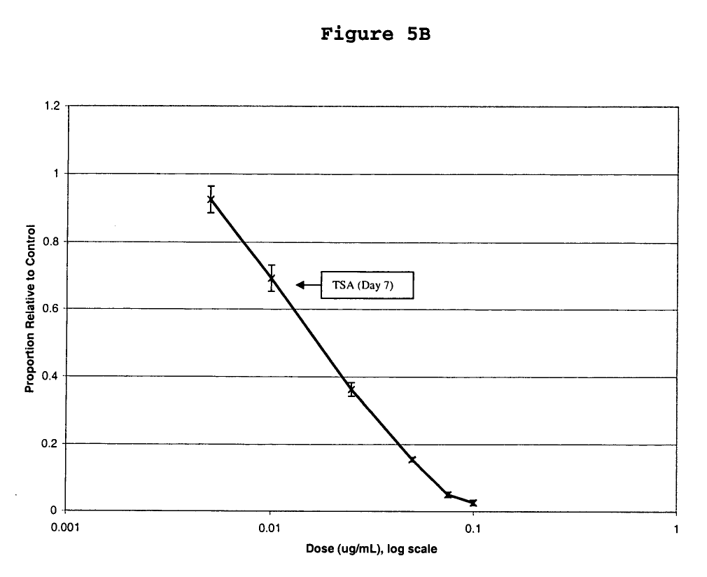

Both valproic acid (Val) (FIG. 5A) and Trichostantin A (TSA) (FIG. 5B) had dose dependent activity as a single agent against U373 cell growth. Although inhibitory doses of valproic acid were in the mM range, the antiepileptic drug is tolerated in humans at serum concentrations approaching 1.0 mM for weeks. Trichostatin A, in contrast, is active at nM concentrations against U373.

Compound LB-1, when combined with Trichostatin A or when combined with 13-cis retinoic acid synergistically inhibited the growth of GBM cell line U373 as shown below in Table 3.

TABLE 3
LB-1 +/− 13-cis Retinoic Acid (CIS-RA) and LB-1 +/− Trichostatin
A (TSA) Inhibition of GBM Cell Line U373

Percent of Control

| | | Expected If |
|---|---|---|
| | Observed | Additive |
| Cis-RA 50 μM | 93.3 +/− 2.2 | |
| TSA 0.033 μM (0.01 μg/ml) | 71.6 +/− 0.4 | |
| LB-1 1 μM | 97.9 +/− 1.0 | |
| LB-1 5 μM | 52.5 +/− 2.9 | |
| Cis-RA 50 μM + LB-1 1 μM | 79.3 +/− 3.2 | 91.3 |
| Cis-RA 50 μM + LB-1 5 μM | 31.6 +/− 2.0 | 49.0 |
| TSA 0.033 μM + LB-1 1 μM | 65.7 +/− 2.0 | 70.1 |
| TSA 0.033 μM + LB-1 5 μM | 13.9 +/− 1.0 | 37.6 |

The two drugs are synergistic in their inhibition of the growth of U373 cells. The percent survival of the cells after exposure to two drugs in combination is less than would be expected from the percent survival of the cells when exposed to each of the two drugs at the same doses used in the combination.

### EXAMPLE 5

### Determination of Tumor Type Specificity

To determine whether there is tumor type specificity of the inhibitory properties of PP2A inhibitors, retinoic acid and Trichostatin A, we measured their inhibitory effects as single agents against the GBM line U373, a breast cancer line, MCF-7 (obtained from ATCC) and a kidney cancer cell line, UMRC (UMRC obtained by Dr. Zhuang, NINDS, NIH from the Intramural Research Support Program, SAIC, National Cancer Institute, Frederick Cancer Research and Development Center).

### Results:

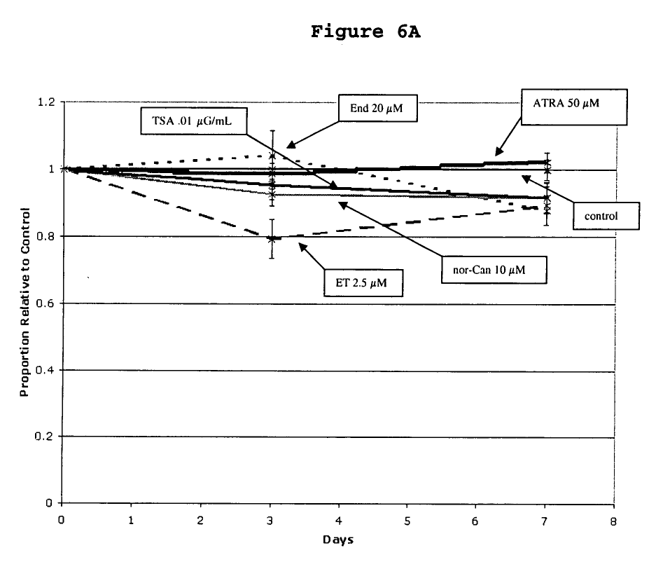

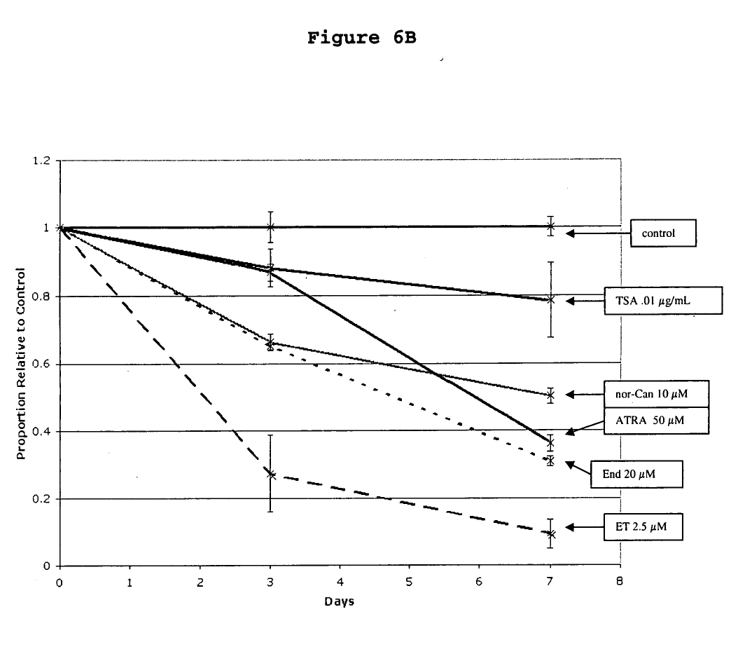

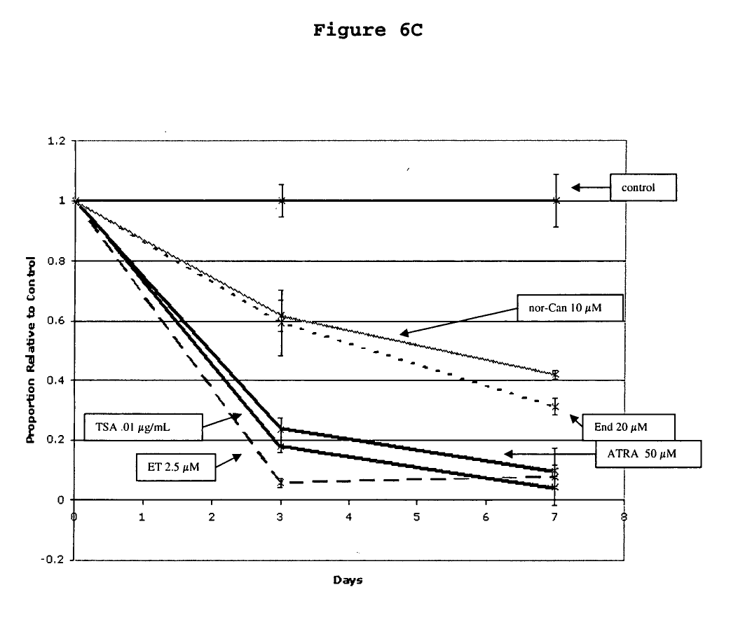

The kidney cancer cell line, UMRC (FIG. 6A) was less sensitive than the brain tumor line, U373 (FIG. 6B) whereas the breast cancer line, MCF-7 (FIG. 6C) was as sensitive as U373 to all-trans retinoic acid, endothal thioanhydride, norcantharidin, endothal, and Trichostatin A. There is some cell type specificity of these drugs for GBMs. The activity of the drugs against MCF-7 cells indicates that regimens being developed for brain tumor treatment may also be useful against breast cancer as well as other tumors that overexpress N—CoR.

### Discussion

To identify novel therapeutic targets, the proteomes of 7 GBM tissues and 7 normal brain tissues were compared using selective microdissection, two dimensional gel electrophoresis (2-DGE) and liquid chromatography-mass spectroscopy (LCMS).

One protein found to have increased expression in GBM as compared to normal brain is nuclear receptor co-repressor (N—CoR), a regulator of the normal neural stem cell pool. (See FIG. 1A) Expression of N—CoR in GBM was confirmed by immunohistochemistry and Western blotting. (See FIGS. 1B and 1C) In contrast, N—CoR was not detectable in normal brain topographically matched to the location of the tumor specimen.

N—CoR is expressed in the nucleus of neural stem cells (NSCs). (Hermanson et al. (2002)) Following phosphatidyl-inositol-3-OH kinase/Akt1 kinase-dependent phosphorylation, N—CoR translocates to the cytoplasm and leads to astrocytic differentiation of NSCs. The nuclear retention of N—CoR, therefore, is essential for the maintenance of NSCs in the undifferentiated state (Hermanson et al.). Analagous to CD133+NSC found within the developing brain, brain tumor stem cells (BTSC) bearing CD133 have been identified within GBM. (Uchida et al. (2000); and Singh et al. (2003)) BTSC are capable of proliferation, self-renewal, and differentiation. BTSC, but not CD133-differentiated tumor cells, are able to recapitulate tumors upon xenograft transplantation. (Singh et al. (2004)).

To characterize a potential role for N—CoR in BTSC differentiation, the relationship between astroglial differentiation within GBMs and N—CoR localization was investigated. Expression of glial fibrillary acidic protein (GFAP), an established marker of astroglial differentiation, and the subcellular localization of N—CoR was assessed by indirect immunofluorescence microscopy on primary cell cultures, established cell lines (A-172, HS683, U87, U251, and U343 MG-A), and frozen and paraffin-embedded tissue sections of GBM. Nuclear expression of N—CoR correlated with the absence of GFAP expression in the cells of primary cultures and tissue sections (see FIG. 1D), whereas cytoplasmic expression of N—CoR correlated with positive expression of GFAP (see FIG. 1D). Some of the tumor cells with nuclear expression of N—CoR also expressed CD133 (see FIG. 1E).

To examine the role of N—CoR in GBM development and differentiation, cultured BTSC isolated from GBM were treated with ciliary neurotrophic factor (CNTF), an agent which has previously been shown to induce the astrocytic differentiation of NSC in vitro. (Hughes et al. (1988)). The cultured BTSC were then assessed for GFAP expression along with subcellular N—CoR localization. Similar to NSC, BTSC cultured with CNTF began to express GFAP (see FIG. 2A). Western blot analysis of the cytoplasmic fraction of CNTF-treated BTSC demonstrates translocation of N—CoR to the cytoplasm (see FIG. 2B). Both cytoplasmic N—CoR and GFAP expression peaked at day 7 following CNTF stimulation.

Retinoids, metabolites of vitamin A, have been examined therapeutically in a variety of tumors, including gliomas. (Yung et al. (1996)) N—CoR is closely associated with the retinoid receptor and is released upon ligand binding to the receptor. (Bastien et al. (2004)) We hypothesized that one effect of retinoids on malignant gliomas may be the induction of differentiation by the binding of retinoids to the retinoid receptor followed by dissociation of the N—CoR/retinoid receptor complex and translocation of N—CoR to the cytoplasm. This idea would explain the previous observation of increase GFAP expression in a glioma cell line (U343 MG-A) treated with retinoids. (Rudka et al. (1988)) To test this we targeted two different sites, individually or simultaneously, within the N—CoR pathway by treating the GBM cell line U343 MG-A with 50 μM of retinoic acid (RA) and/or 10 nM of okadaic acid (OA), a protein phosphatase-1 and protein phosphatase-2A inhibitor. By preventing the action of protein phosphatase-1 and protein phosphatase-2A, okadaic acid increases the phosphorylated form of N—CoR and promotes its subsequent cytoplasmic translocation. (Hermanson et al. (2002))

Cell counts monotonically increased for OA, RA, and controls through day 16 (see FIG. 2C). Retinoic acid and low-dose okadaic acid each had a modest effect on cell growth. Combination of retinoic acid and okadaic acid shows synergistic reduction in cell growth.

These observations demonstrate a role for N—CoR in BTSC differentiation and suggest a new treatment paradigm for glioblastoma multiforme. To our knowledge, this is the first example of a therapeutic strategy directly targeting BTSCs. Differentiation and growth inhibition of BTSCs is achieved by the synergistic combination of two compounds acting at different levels of the N—CoR pathway.

Several molecules, including okadaic acid, that have anti-PP2A activity synergize with all-trans retinoic acid and 13-cis retinoic acid in inhibiting the growth of GBM cells in vitro. The most effective group of phosphatase inhibitors synergizing with retinoic acids that have been evaluated are analogs of the ancient therapeutic agent, mylabris, derived from the crushed bodies of the blister beetle, in which the principal active agent is cantharidin, a known potent inhibitor of PP2A (Wang, 1989; Peng et al., 2002).

Cantharidin has anti-tumor activity against human cancers of the liver (hepatomas) and of the upper gastrointestinal tract but is toxic to the urinary tract (Wang, 1989). Norcantharidin, a demethylated cantharidin, maintains antitumor activity of cantharidin against hepatomas and cancers of the stomach and esophagus, but has little or no urinary tract toxicity. Norcantharidin increased the life span of 244 patients with primary hepatoma from 4.7 to 11.1 months and increased 1-year survival from 17% to 30% compared to historical control patients treated with standard chemotherapy. Norcantharidin also stimulates white blood cell production in patients and mice, a phenomenon not understood mechanistically, but a pharmacological effect of potential benefit as an anticancer agent (Wang et al., 1986; Wang, 1989).

In the past, several cantharidin analogs had been synthesized and evaluated for anti-phosphatase activity and for their ability to inhibit the growth of cancer cells in culture (Sakoff and McClusky, 2004; Hart et al.; 2004). Some of the previously evaluated modified norcantharidin molecules inhibited the growth of several human tumor cell lines. The activity of norcantharidin analogs against GBMs or the activity of norcantharidins combined with other potential anti-tumor agents was not analyzed. Further studies included 16 “modified norcantharidins” evaluated for activity against four human tumor cell lines including ovarian, kidney, colorectal and lung as well as a mouse leukemia line. None were as active as single agents as cantharidin or norcantharidin and none were evaluated for activity in combination with another antitumor agent (McCluskey et al., US Serial No. 2006/0030616, 2006).

A different series of cantharidin analogs had been previously synthesized and evaluated as pesticides and for antitumor activity against cancer cell lines. The dicarboxylic acid derivative of norcantharidin, endothal, was developed as an herbicide and defoliant. Forty-three analogs of endothal and cantharidin have been developed and assessed for their activity as herbicides and their lethality to mice (Matsuzawa et al., 1987). Endothal thioanhydride was shown to be a more potent insecticide than endothal but was toxic to the liver of mice (Matsuzawa et al., 1987; Kawamura et al., 1990).

Endothal and endothal thioanhydride, like cantharidin, inhibit the activity of PP2A and to some extent, the activity of PP1 (Erdodi et al., 1995). In cell lysates the order of the potency of inhibition of PP2A was cantharidin, endothal and endothal thioanhydride. In the liver of the intact animal, the potency of the inhibition of PP2A was endothal thioanhydride, followed by cantharidin and endothal. The differences depended on the lipid solubility of the compounds (Erdodi, 1995). Endothal thioanhydride is highly lipid soluble and insoluble in water and is the most toxic in vivo, whereas endothal is highly water soluble and is the least toxic, with norcantharidin falling in between with respect to lipid solubility and toxicity.

To identify novel therapeutic targets for the treatment of glioblastoma multiforme (GBM), compound LB-1, norcantharidin (nor-Can), endothal (End), and endothal thioanhydride (ET) were evaluated for their ability to inhibit glioblastoma multiforme.

Each of the norcantharidin analogs inhibited the growth of GBMs in a dose dependent manner in vivo as shown in FIG. 3.

From graphic plots of the GBM cell line U373 as a function of exposure to different doses of drug for 7 days, the concentration of each compound that inhibited brain tumor cell proliferation by 50% (IC50) was estimated. The IC50s expressed in micro-molarity (μM), were: 2.5, 3.0, 12.0, and 15.0 for endothal thioanhydride, compound LB-1, norcantharidin, and endothal respectively as seen in FIG. 3.

We found that, on a molar basis, of the phosphatase inhibitors tested, endothal thioanhydride was the most potent inhibitor of GBMs in vitro compared to norcantharidin and endothal.

In combination with anti-phosphatases, retinoids synergistically inhibit the proliferation of glioblastoma multiforme. Synergism (potentiation) of the inhibitory activity of two drugs in combination is said to be present when the percent survival in the presence of two drugs is less than the product of the percent survivals of the two drugs used alone at the same doses in the combination.

The inhibitory activity of retinoids was further evaluated in combination with endothal as well as individually. As shown in FIG. 4A, increase in the dose of ATRA exhibits inhibitory activity on glial cancer cells. However, as demonstrated in FIG. 4B, the combination of endothal with ATRA demonstrated a synergistic reduction in cell growth.

The expected percent survival of U373 cells exposed to the combination of ATRA and End is 50% (77% by ATRA×65% by End=50%) whereas the observed survival was 32%. The expected percent survival in the presence of the combination of ATRA and LB-1 is 60% (77% by ATRA×78% by LB-1=60%), whereas the observed survival was 53%.

Endothal combined with 13-cis retinoic acid (cis-RA) synergistically inhibits U373 cell growth over a period of 7 days as compared to the more modest inhibition when endothal was used individually as seen in FIG. 4C.

The presence of 13-cis retinoic acid at 50 uM had little effect when combined with endothal at 1.0 μM (96%). At higher doses of endothal, simultaneous exposure to the same amount of 13-cis retinoic acid decreased cell survival from 96% at 5 uM endothal alone to 80% in combination with 13-cis retinoic acid and from 76% survival at 10 uM endothal alone to 53% in combination with 13-cis retinoic acid.

Trichostatin A is a natural product extracted from streptomyces, which has anti-fungal and anti-cancer activity in vitro and in human cancer xenografts (Yoshida et al., 1990; Sanderson et al., 2004). Valproic acid is a widely used anti-seizure medicine that inhibits human cancer cells in vitro at concentrations achievable in the plasma of humans (Göttlicher et al., 2001; Blaheta et al., 2002). As demonstrated in FIGS. 5A and 5C, both valproic acid (Val) and Trichostatin A (TSA) had dose dependent activity as single agents against U373 cell growth. Although inhibitory doses of valproic acid were in the mM range, the drug is tolerated in humans at serum concentrations approaching 1.0 mM for weeks. Trichostatin A, by contrast, is active at nM concentrations against U373. Given the low toxicity in non-pregnant adults, both compounds combined with endothal could be potentially effective regimens in the treatment of GBM in humans.

Cantharidin homologs and okadaic acid act synergistically when administered with valproic acid or trichostatin A to inhibit growth of GBM cells. Both valproic acid and trichostatin A are known to have anti-histone deacetylase (HDAC) activity.

To determine whether there is tumor type specificity of the inhibitory properties of PP2A inhibitors, retinoic acid and Trichostatin A we measured their inhibitory effects as single agents against the GBM line U373, a breast cancer line, MCF-7 (obtained from ATCC) and a kidney cancer cell line, UMRC (UMRC obtained by Dr. Zhuang, NINDS, NIH from the Intramural Research Support Program, SAIC, National Cancer Institute, Frederick Cancer Research and Development Center).

The kidney cancer cell line, UMRC (FIG. 6A) was less sensitive than the brain tumor line, U373 (FIG. 6B) whereas the breast cancer line, MCF-7 (FIG. 6C) was as sensitive as U373 to all-trans retinoic acid, endothal thioanhydride, norcantharidin, endothal, and Trichostatin A. There is some cell type specificity of these drugs for GBMs. The activity of the drugs against MCF-7 cells indicates that regimens being developed for brain tumor treatment are likely also useful against breast cancer and other tumors that overexpress N—CoR.

### REFERENCES

1. Bastien et al. (2004), Gene, Vol. 328, pp. 1-16.
2. Blaheta, A et al. (2002), “Valproate and Valproate-Analogues: potent Tools to Fight Against Cancer,” Current Medicinal Chemistry, Vol. 9, pp. 1417-1433.
3. Drewinko et al. (1967), “Combination chemotherapy in vitro with adriamycin. Observations of additive, antagonistic, and synergistic effects when used in two-drug combinations on cultured human lymphoma cells,” Cancer Biochem. Biophys., Vol. 1, pp. 187-195.
4. Erdodi et al. (1995), “Endothal thioanhydride inhibits proteins phosphatases-1 and -2A inhibition, and anticancer activity,” Am. J. Physiol. (Cell Physiol.) Vol. 38, pp. C1176-1184.
5. Giannini, R. and Cavallini, A. (2005), “Expression analysis of a subset of coregulators and three nuclear receptors in colorectal carcinoma.” Anticancer Research, Vol. 36, No. 6B, pp. 4287-4292.
6. Göttlicher, M et al. (2001), “Valproic acid defines a novel class of HDAC inhibitors inducing differentiation of transformed cells,” Eur. Mol. Bio. Journal, Vol. 20, no. 24, pp. 6969-6978.
7. Hart, M E et al. (2004), “Modified norcantharidine: synthesis, protein phosphatases 1 and 2A inhibition, and anticancer activity,” Bioorganic & Medicinal Chemistry Letters, Vol. 14, pp. 1969-1973.
8. Havrilesky, L J et al. (2001), “Relationship between expression of coactivators and corepressors of hormone receptors and resistance of ovarian cancers to growth regulation by steroid hormones,” J. Soc. Gynecol. Investig., Vol. 8, pp. 104-113.
9. Hermanson et al. (2002), “N—CoR controls differentiation of neural stem cells into astrocytes,” Nature, Vol. 419, pp. 934-939.
10. Hughes et al. (1988), Nature, Vol. 335, pp. 70-73.
11. Kawamura, N. et al. (1990), “Endothall Thioanhydride: Structural Aspects of Unusually High Mouse toxicity and Specific Binding Site in Liver,” Chem. Res. Toxicol., Vol. 3, pp. 318-324.
12. Kovach, J S et al. (1985), “Enhancement of the antiproliferative activity of human interferon by polyamine depletion,” Cancer Treat. Rep., Vol. 69, pp. 97-103.
13. Matsuzawa, M. et al. (1987), “Endothal and Cantharidin Analogues: Relation of Structure to Herbicidal Activity and Mammalian Toxicity,” J. Agric. Food Chem., Col. 35, No. 5.
14. Momparlet, R L. (1980), “In vitro systems for evaluation of combination chemotherapy,” Pharmacol. Ther., Vol. 8, pp. 21-35.
15. Myers, E. et al. (2005), “Associations and Interactions Between Ets-1 and Ets-2 and Coregulatory Proteins, SRC-1, AIB1 and NCoR in Breast Cancer,” Clin. Cancer Res., Vol. 11, pp. 2111-2122.
16. Park, D M. et al., “N—CoR pathway targeting induces glioblastoma derived cancer stem cell differentiation, Cell Cycle, in press.
17. Peng, F. et al. (2002), “Induction of apoptosis by norcantharidin in human colorectal cell lines: involvement of the CD95 receptor/ligand,” J. Cancer Res. Clin. Oncol., Vol. 128, pp. 223-230.
18. Rutka et al. (1988), “Effect of retinoids on the proliferation, morphology and expression of glial fibrillary acidic protein of an anaplastic astrocytoma cell line,” Int. J. Cancer, Vol. 42, pp. 419-427.
19. Sanderson, L et al. (2004), “Plasma Pharmacokinetics and Metabolism of the Histone Deacetylase Inhibitor Trichostatin A after Intraperitoneal Administration to Mice,” Drug Metabolism and Disposition, Vol. 32, No. 10, pp. 1132-1138.
20. Sakoff, J A. (2004), “Protein Phosphatase Inhibition: Structure Based Design, Towards New Therapeutic Agents,” Current Pharmaceutical Design, Vol. 10, pp. 1139-1159.
21. Singh et al. (2003), “Identification of a cancer stem cell in human brain tumors,” Cancer Research, Vol. 63, pp. 5821-5828.
22. Singh et al. (2004), “Identification of human brain tumour initiating cells,” Nature, Vol. 432, pp. 396-401.
23. Stupp et al. (2005), N. Engl. J. Med., Vol. 352, pp. 987-996.
24. Uchida et al. (2000), Proc. Natl. Acad. Sci. USA, Vol. 97, pp. 14720-14725.
25. U.S. Pat. No. 6,949,624, Liu et al.
26. U.S. Patent Publication No. 2004/0197888, Armour et al.
27. U.S. Patent Publication No. 2004/0253637, Buechler et al.
28. U.S. Patent Publication No. 2005/0203082, Hsu et al.
29. U.S. Patent Application No. 2006/0030616, McCluskey et al.
30. Valeriote, F., (1975), “Synergistic interaction of anticancer agents: A cellular perspective,” Cancer Chemother. Rep., Vol. 59, pp. 895-900.
31. Wang, G S (1983), “Hydrolysis and demethylation of cantharidin on the relief of its urinary irritation,” Chin. Pharmac. Bull., Col. 18, pp. 18-19.
32. Wang, G S (1989), “Medical uses of mylabris in ancient China and recent studies,” J. Ethnopharmacol., Vol. 26, pp. 147-162.
33. Wang, G S et al. (1986), “Results of clinical trials in 244 cases of primary hepatoma and with norcantharidin,” Chinese. Pharm. Bull., Vol. 21, pp. 90-93.
34. Wang, G S et al. (1987), “Effect of norcantharidin on the number of white blood cells,” Chinese Pharm. Bull., Vol. 22, pp. 517-519.
35. Waters, C E et al. (2004), “Analysis of co-factor function in glucocorticoid-resistant small cell carcinoma line,” J. Endocrinol., Vol. 183, pp. 375-383.
36. Yoshida, M et al. (1990), “Potent and Specific Inhibition of Mammalian Histone Deacetylase Both in vivo and in vitro by Trichostantin A,” Journal of Biological Chem., Vol. 265, No. 28, pp. 17174-17179.
37. Yung et al. (1996) Clin. Cancer Res. Vol. 2, pp. 1931-1935.

1. A method of treating a patient suffering from a tumor overexpressing N—CoR comprising administering to the patient a phosphatase ligand in an amount effective to treat the patient.

2. The method of claim 1 further comprising administering to the patient a retinoid receptor ligand in an amount such that the amount of each of the phosphatase ligand and the retinoid receptor ligand is effective to treat the patient.

3. The method of claim 1 further comprising administering to the patient a histone deacetylase ligand in an amount such that the amount of each of the phosphatase ligand and the histone deacetylase ligand is effective to treat the patient.

4. The method of claim 1 further comprising administering both a retinoid receptor ligand and a histone deacetylase ligand each in an amount such that the amount of each of the phosphatase ligand, the histone deacetylase ligand and the retinoid receptor ligand is effective to treat the patient.

5. The method of claim 1, wherein the phosphatase ligand is a protein phosphatase inhibitor.

6. The method of claim 1, wherein the phosphatase ligand is selected from the group consisting of 1-nor-okadaone, antimonyl tartrate, bioallethrin, calcineurin, cantharidic acid, cantharidin, calyculin, cypermethrin, DARPP-32, deamidine, deltamethrin, diaminopyrroloquinazolines, endothal, endothal thioanhydride, fenvalerate, fostriecin, imidazoles, ketoconazole, L-4-bromotetramisole, levamisole, microcystin LA, microcystin LR, microcystin LW, microcystin RR, molybdate salts, okadaic acid, okadol, norcantharidin, pentamidine, pentavalent antimonials, permethrin, phenylarsine oxide, phloridzin, protein phosphatase inhibitor-1 (I-1), protein phosphatase inhibitor-2 (I-2)pyrophosphate, salubrinal, sodium fluoride, sodium orthovanadate, sodium stibogluconate, tartrate salts, tautomycin, tetramisole, thrysiferyl-23-acetate, vanadate, vanadium salts and antileishmaniasis compounds, including suramin and analogues thereof.

7. The method of claim 3, wherein the histone deacetylase ligand is an inhibitor.

8. The method of claim 7, wherein the inhibitor is HDAC-3 (histone deacetylase 3).

9. The method of claim 3, wherein the histone deacetylase ligand is selected from the group consisting of 2-amino-8-oxo-9,10-epoxy-decanoyl, 3-(4-aroyl-1H-pyrrol-2-yl)-N-hydroxy-2-propenamide, APHA Compound 8, apicidin, arginine butyrate, butyric acid, depsipeptide, depudecin, HDAC-3, m-carboxycinnamic acid bis-hydroxamide, N-(2-aminophenyl)-4-[N-(pyridin-3-ylmethoxycarbonyl)aminomethyl]benzamide, MS 275, oxamfiatin, phenylbutyrate, pyroxamide, scriptaid, sirtinol, sodium butyrate, suberic bishydroxamic acid, suberoylanilide hydroxamic acid, trichostatin A, trapoxin A, trapoxin B and valproic acid.

10. The method of claim 1, wherein the tumor overexpressing N—CoR is glioblastoma multiforme, breast cancer, colorectal cancer, small cell lung cancer or ovarian cancer.

11. The method of claim 10, wherein the tumor overexpressing N—CoR is breast cancer.

12. A method of inhibiting growth of a tumor overexpressing N—CoR in a patient comprising administering to the patient a phosphatase ligand in an amount effective to affect N—CoR so as to induce differentiation of cells of the tumor overexpressing N—CoR and inhibit growth of the tumor in the patient.

13. The method of claim 12, further comprising administering to the patient a retinoid receptor ligand in an amount such that the amount of each of the phosphatase ligand and the retinoid receptor ligand is effective to affect N—CoR so as to induce differentiation of cells of the tumor overexpressing N—CoR and inhibit growth of the tumor in the patient.

14. The method of claim 12, further comprising administering to the patient a histone deacetylase ligand in an amount such that the amount of each of the phosphatase ligand and the histone deacetylase ligand is effective to affect N—CoR so as to induce differentiation of cells of the tumor overexpressing N—CoR and inhibit growth of the tumor in the patient.

15. The method of claim 12, further comprising administering to the patient both a retinoid receptor ligand and a histone deacetylase ligand, each in an amount such that the amount of each of the phosphatase ligand, the histone deacetylase ligand and the retinoid receptor ligand is effective to affect N—CoR so as to induce differentiation of cells of a tumor overexpressing N—CoR and inhibit growth of the tumor in the patient.

16. The method of claim 12, wherein the tumor overexpressing N—CoR is glioblastoma multiforme, breast cancer, colorectal cancer, small cell lung cancer or ovarian cancer.

17. (canceled)

18. A method of identifying a compound or a mixture of compounds capable of inducing differentiation or inhibiting proliferation of cells of a tumor overexpressing N—CoR comprising:
(a) culturing a first population of specified human cells in the absence of the compound or the mixture of compounds in both serum and serum free conditions;
(b) separately culturing a second population of such human cells in the presence of the compound or the mixture of compounds;
(c) comparing the rate of growth of the cultured human cells in step (a) with the rate of growth of the cultured human cells in step (b);
(d) identifying the compound or the mixture of compounds which inhibited, or reduced the rate of, growth of the cultured human cells in step (b) as compared to the rate of growth of the cultured human cells in step (a); and
(e) measuring the level of N—CoR in the cytoplasm and in the nucleus of the cultured human cells from step (b) whose growth was inhibited or whose rate of growth was reduced in the presence of the compound or the mixture of compounds with the levels of N—CoR in the cultured human cells from step (a), wherein the presence in the sample of decreased levels of N—CoR indicates that the compound or the mixture of compounds is capable of inducing differentiation or inhibiting proliferation of cells of tumors overexpressing N—CoR, so as to thereby identify the compound or the mixture of compounds.

19-22. (canceled)

23. A method of determining the likelihood of successfully treating a subject suffering from a tumor overexpressing N—CoR:
a) obtaining a sample from the subject containing cells of a tumor overexpressing N—CoR; and
b) measuring the level of N—CoR in the cytoplasm and in the nucleus of cells in the sample so obtained,
wherein the presence in the sample of increased levels of N—CoR in the nucleus and a decreased level of N—CoR in the cytoplasm of the cells indicates that there is a greater likelihood of successfully treating the subject.

24-27. (canceled)

28. A method of assessing the likelihood that a patient previously suffering from and treated for a tumor overexpressing N—CoR has suffered a recurrence of such tumor which comprises:
a) obtaining a serum sample from the subject; and
b) measuring the level of N—CoR in the serum sample so obtained;
wherein the presence in the serum sample of increased levels of N—CoR relative to a previous level of N—CoR indicates that the patient is likely suffering from a recurrence of a tumor overexpressing N—CoR.

29-30. (canceled)

31. A method of assessing the likelihood that a patient is suffering from a tumor overexpressing N—CoR which comprises:
a) obtaining a serum sample from the subject; and
b) measuring the level of N—CoR in the serum sample so obtained;
wherein the presence in the serum sample of increased levels of N—CoR relative to a normal reference standard indicates that the patient is likely suffering from a tumor overexpressing N—CoR.

32-87. (canceled)
